(19) United States
(12) Patent Application Publication
(10) Pub. No.: US 2014/0294671 A1
(43) Pub. Date: Oct. 2, 2014

(54) CARDIOPULMONARY BYPASS

(71) Applicant: Basildon and Thurrock University Hospitals NHS Foundation Trust, Basildon, Essex (GB)

(72) Inventors: John Wallace Mulholland, London (GB); Paul Joseph Malone, Westcliff-on-Sea (GB); Gary Christian Cordery, Prittlewell (GB)

(21) Appl. No.: 14/354,533

(22) PCT Filed: Oct. 25, 2012
(86) PCT No.: PCT/GB2012/052650

§ 371 Date: Apr. 25, 2014

(30) Foreign Application Priority Data
Oct. 27, 2011 (GB) 1118595.6

(51) Int. Cl. A61M 1/36

(52) U.S. Cl. CPC A61M 1/3627; A61M 1/3666
USPC 422/44

(57) ABSTRACT

The invention provides an improved apparatus and methods for carrying out cardiopulmonary bypass. In particular, the invention relates to pericardial blood suction apparatus and methods, which can be carried out during cardiopulmonary bypass procedures.

The present invention relates to cardiopulmonary bypass, and to improved apparatus and methods for carrying out cardiopulmonary bypass. In particular, the invention relates to pericardial blood suction apparatus and methods, which can be carried out during cardiopulmonary bypass procedures.

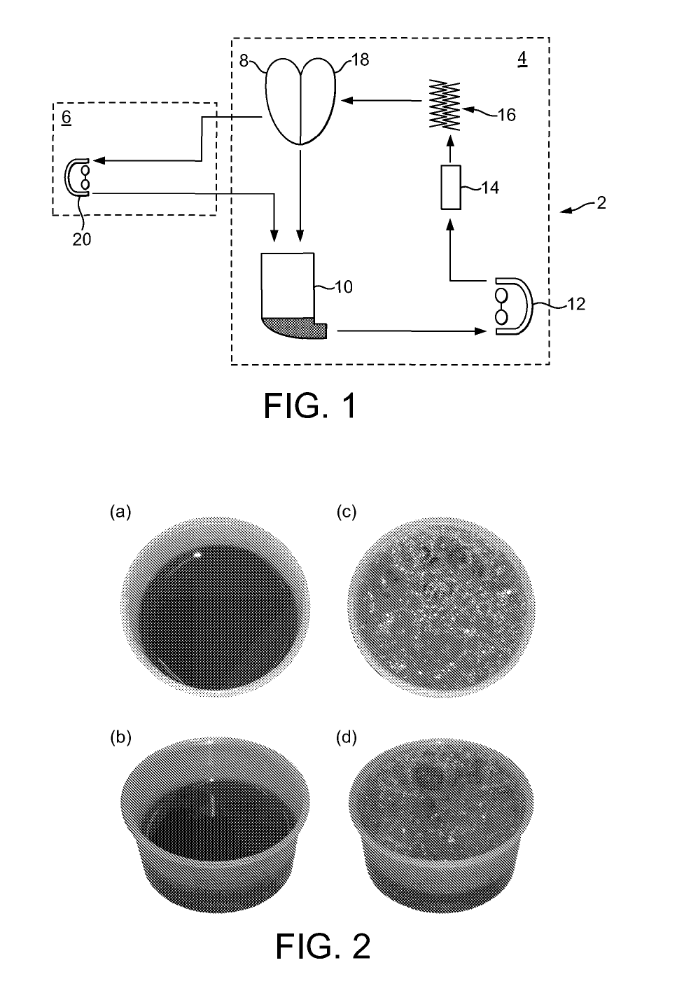

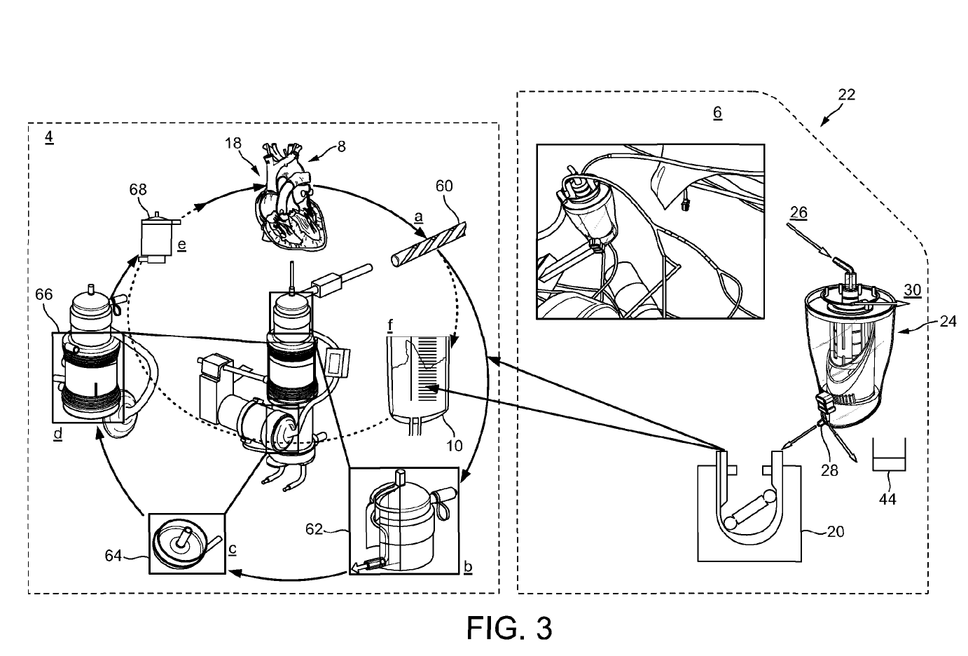

Artificial heart and lung support, also known as cardiopulmonary bypass (CPB), is required to perform most cardiac surgical operations. As illustrated in FIG. 1, a “conventional” CPB apparatus 2 is shown consisting of a primary system 4 and secondary system 6. The primary system 4 is systemic, and forms the artificial heart and lung support system in which the patient's blood is drained either via gravity or assisted drainage out of the right atrium of the heart 8 into the venous reservoir 10. It is then pumped, by a roller pump 12, into a heat exchanger 14 and an artificial lung (also known as an oxygenator) 16, and then eventually returned back into the patient via the aorta 18. From the right atrium, the blood is initially fed into a venous reservoir 10, which is placed in series in the primary system 4, and which acts as a capacitance chamber, where the blood is filtered and can be mixed with fluids and any required drugs. It is then passed to a heat exchanger 14, which allows the patient's core temperature to be varied, and then fed to the artificial lung (or oxygenator) 16 where oxygen is added to the blood and carbon dioxide is removed, prior to the blood passing through a filter and ultimately being returned to the heart 8 via the aorta 18. As illustrated in FIG. 3, a “closed circuit” CPB apparatus 2 is shown which also consists of a primary system 4, except that in this set-up, the venous reservoir 10 is placed in parallel.

The primary system of “conventional” CPB apparatus, therefore, essentially replicates the function of the heart and lungs. The secondary system 6, on the other hand, is designed to remove blood from the patient's pericardium (i.e. the chest cavity) via a low pressure suction (LPS) device and, where appropriate, from the heart chambers via a vent system. A roller pump 20 is used to remove the blood from the patient, before returning it back to the series venous reservoir 10 of the primary system 4 of CPB. The LPS and vent system is therefore a convenient way of conserving blood (i.e. returning it to the primary systemic system) while keeping the operating field of the patient's chest cavity clear of blood to improve the visibility for the surgeon during the operation, before feeding it back into the primary system, where the volume is required to maintain the heart and lung support.

Therefore, CPB is a form of extracorporeal circulation, temporarily taking over the function of the heart and lungs during surgery, maintaining the circulation of blood and the oxygen content of the body, while simultaneously keeping the surgeon's operating field (i.e. the pericardium) clear. The CPB pump itself is often referred to as a heart-lung machine or “the pump”, and is operated by a Perfusionist in association with surgeons who connect the pump to the patient's body.

One problem with CPB, however, is that it is an extremely non-physiological procedure, involving the blood having to pass along non-physiological geometries, and along lengths of silicone and PVC tubing, and so is very damaging to the patient's blood. One of the most blood-damaging aspects of CPB is the secondary LPS system, which comprises a first length of ¼″ PVC tubing extending from the patient's pericardium to the roller pump, and then a second piece of tubing extending from the roller pump to the venous reservoir of the primary CPB circuit. The LPS system exposes the patient's blood not only to negative pressures, but also results in the patient's blood interfacing, and therefore mixing, with air, thereby creating frothy blood, as is clearly shown in FIGS. 2(c) and 2(d). These mechanisms are well-documented to cause significant damage to the blood, especially to the red and white blood cells, and platelets, reducing the blood's capacity for carrying oxygen and healing, and the patient's ability to fight infection.

Another problem presented by the LPS system is when a “closed circuit” CPB system is used (as shown in FIG. 3), and the reservoir 10 is in parallel to the artificial heart and lung system. In a closed system, blood is removed kinetically from the right atrium of the heart 8 using a pump 64 via a small filter 62, a heat exchanger (not shown) and oxygenator 66, then through a filter 68 and eventually back to the patient via the aorta 18. As can be seen in FIG. 3, the reservoir 10 sits parallel to this system, which reduces the blood non-physiological surface exposure, as blood does not pass through the reservoir 10 every cycle. However, in this arrangement, the blood returning to the reservoir 10 from the LPS and Vent (i.e. the secondary) system 6 does not automatically enter the primary, systemic system 4, as it would with a reservoir 10 arranged in series. This creates an extra step that needs to be performed during what is already a very complex procedure to manage. Furthermore, if a soft-shell reservoir 10 is used in parallel, such as the one shown in FIG. 3, another extra step is created as the medical practitioner has to constantly stand by the bag throughout the whole procedure and manually de-air the bag of the air that is introduced by the LPS and Vent system. This is achieved by opening a valve on the soft-shell reservoir 10, and physically squeezing the air out. There is therefore a balance between more physiological artificial heart and lung support and increased workload for the medical practitioner. Accordingly, in view of these problems, there is a need for an improved CPB system, and in particular, an improved secondary LPS system.

Thus, according to a first aspect of the invention, there is provided a pericardial blood suction apparatus for use in cardiopulmonary bypass (CPB), wherein the apparatus comprises means for withdrawing blood from a subject's chest cavity and/or heart chamber, and a reservoir in which withdrawn blood is de-aired.

The pericardial blood suction apparatus of the invention can be referred to as a low pressure suction (LPS) and/or vent system (i.e. the secondary system) of CPB, and is capable of conserving blood and keeping the operating field of the chest cavity (i.e. the pericardium) substantially clear of blood during an operation.

Advantageously, unlike prior art secondary (LPS) systems of CPB, which use a roller pump to withdraw blood from a subject's chest cavity and immediately return it back to the primary system of the CPB, the apparatus of the invention includes the reservoir in which the withdrawn LPS and vent blood is temporarily retained, and allowed to settle, thereby resulting in effective and automatic blood de-airing, before it is then returned to the primary system of the CPB, or to a holding reservoir. Accordingly, preferably the apparatus automatically de-airs the blood in the reservoir. Because the blood is automatically de-aired, no extra clinician is needed, thereby reducing the workload of the medical practitioners. The apparatus may return the de-aired blood either automatically or manually to the systemic (i.e. the primary) system of CPB system, or a parallel reservoir. In one embodiment, the blood may be returned to a systemic CPB system post-venous reservoir in the conventional system. Advantageously, the de-aired blood can be automatically returned from the secondary system to the primary system or holding reservoir, thereby reducing blood/air interface.

Surprisingly, the inventors have found that effective de-airing of the withdrawn blood occurs automatically inside the reservoir, and so is a significant improvement of the current use, in “closed circuit” CPB, of a soft-shell reservoir, which must be constantly opened to allow the air to be squeezed therethrough, before the de-aired blood can be fed back to the CPB system, or to a holding reservoir (depending on clinical conditions).

As shown clearly in FIGS. 2(a) and 2(b), improved blood and air management means that no froth forms in the withdrawn blood contained within the reservoir, before it is fed back into the primary CPB system. This is in stark contrast to the frothy condition of the blood that is created using prior art LPS systems, as shown in FIGS. 2(c) and 2(d). Furthermore, advantageously the blood is de-aired sooner in the CPB blood pathway compared to using a known CPB system.

The volume of the reservoir may be between approximately 10 ml and 500 ml, or between 15 ml and 250 ml. The reservoir may comprise a blood inlet through which withdrawn blood is fed into the reservoir, and a blood outlet through which de-aired blood may exit the reservoir. The inlet may be disposed at least adjacent an upper portion of the reservoir. The outlet may be disposed at least adjacent a lower portion of the reservoir. The reservoir may comprise an internal chamber which, in use, contains the withdrawn blood for sufficient time to allow de-airing to occur. By way of example, withdrawn blood may have a residence time within the reservoir of between approximately 1 and 20 seconds, or between approximately 2 and 10 seconds.

The means for withdrawing the blood from the subject may comprise engagement means which is capable of engaging with the subject's chest cavity or pericardium, the engagement means being in fluid communication with the inlet, preferably via a conduit. For example, the engagement means may comprise any kind of suction means such as a suction catheter, a cannula, a yanker sucker, a wand or the like.

The means for withdrawing the subject's blood may comprise a source of negative pressure, i.e. suction pressure. For example, the means for withdrawing the blood may be adapted to create a negative pressure in the reservoir of at least −1 to −120 mm Mercury, preferably −5 to −20 mm Mercury. Preferably, the means for withdrawing the blood may be adapted to create a negative pressure in the reservoir which does not exceed −120 mm Mercury.

The means for withdrawing the blood may be a pump connected to the reservoir. Preferably, the means for withdrawing the blood from the chest cavity and/or heart chamber is a vacuum source. Once the blood has been withdrawn into the reservoir, it is then allowed to settle so that air can escape. The reservoir may be connected to the atmospheric pressure to allow the air to escape.

The pericardial blood suction apparatus may comprise feed means for feeding blood from the reservoir to the subject, the primary CPB system or a holding chamber (such as a capacitance reservoir arranged in parallel), preferably via the outlet, preferably via a conduit. The feed means may comprise a pump, for example a roller pump. The reservoir may comprise a blood sensor, which is adapted, in use, to monitor the volume of blood in the reservoir and control the feed means depending on the blood volume. Preferably, in use, as the volume of blood reaches an upper preset level within the reservoir, the blood level sensor is capable of switching the pump on, to thereby pump blood out of the reservoir, and as the volume of blood reaches a lower preset level within the reservoir, the sensor is capable of switching the pump off, to thereby prevent blood from being pumped out of the reservoir. Preferably, the steady state volume of withdrawn blood contained within the reservoir is between about 10 ml and 500 ml, or between about 15 ml and 25 ml.

The pericardial blood suction apparatus may comprise pressure control means adapted, in use, to control the pressure within the reservoir, and automatically regulate it about a desired preset value. For example, in one embodiment, the preset value of pressure in the reservoir may be approximately or between ‘5 and −25 mm, or between about −10 and −20 mm Mercury, or between about −13 and −17 mm Mercury. The pressure control means may comprise a pressure transducer which is adapted, in use, to detect the pressure inside reservoir. The pressure transducer may be connected to the means for withdrawing blood from the subject, preferably via an air inlet thereof.

Preferably, the pressure control means comprises a valve, which valve, when open, connects the reservoir to atmospheric pressure. Preferably, the pressure control means comprises a control circuit, which is adapted to control the opening and closing of the valve depending on the pressure within the reservoir. The valve may be a controllable valve. In one embodiment, the valve may be a solenoid valve. The pressure transducer may be connected to an amplifier circuit, which is capable of generating an output which may be fed to an input of a processor. The processor may be adapted to receive the input signal and capable of creating an output to open or close the valve, depending on the pressure level inside the reservoir. It will be appreciated that opening the valve may allow the internal pressure in the reservoir to be equalised with atmospheric pressure until the internal pressure equals the desired preset level. The pressure control means may be adapted, in use, to close the valve, when the pressure inside the reservoir reaches its preset level. Advantageously, this process of opening and closing the valve loops continually and automatically, and ensures that the pressure in the reservoir is maintained about the preset fixed point.

In a second aspect, there is provided a cardiopulmonary bypass system (CPB) comprising the pericardial blood suction apparatus according to the first aspect.

It will be appreciated that the pericardial blood suction apparatus constitutes the secondary LPS and/or vent system of the CPB system. Preferably, therefore, the CPB system of the second aspect also comprises a primary or systemic system. The primary system may comprise means for removing blood from the subject's heart, preferably the right atrium. The means for removing blood may be a pump, preferably a roller pump. The primary system may comprise a venous reservoir in which removed blood may be contained. The venous reservoir may be placed in series or in parallel in the primary system. The venous reservoir may be an ‘open to air’ hard-shell reservoir or a ‘closed to air’ soft-shell reservoir. Thus, the CPB system may comprise a hard-shell reservoir arranged in series or in parallel. The CPB system may alternatively comprise a soft-shell reservoir arranged in series or in parallel. The CPB system of the second aspect may be a conventional circuit system or a closed circuit system.

The venous reservoir may comprise means for filtering the removed blood. The primary system may comprise a heat exchanger for varying the temperature of the removed blood. The primary system may further comprise an oxygenator for adding oxygen to the removed blood and/or removing carbon dioxide from the blood. The primary system may be adapted, in use, to remove blood from the right atrium of a subject's heart, and return it to the subject via the aorta.

As shown in FIGS. 2(a) and (b), the inventors have clearly demonstrated that the apparatus of the invention effectively reduces the extent of blood/air mixing and damage that is caused to blood withdrawn from the subject's chest cavity.

Hence, in a third aspect, there is provided use of the pericardial blood suction apparatus according to the first aspect, or the cardiopulmonary bypass system (CPB) of the second aspect, for reducing damage caused to blood withdrawn from a subject's pericardium and/or heart chamber.

In a fourth aspect, there is provided a method of reducing damage caused to blood withdrawn from a subject's pericardium and/or heart chamber, the method comprising:
(i) withdrawing blood from a subject's pericardium and/or heart chamber, and
(ii) feeding the withdrawn blood into a reservoir, where the blood is de-aired.

The use of the third aspect and method of the fourth aspect reduce damage to the red and white blood cells, and platelets, which would otherwise reduce the blood's capacity for carrying oxygen and causing clots. Accordingly, the subject's ability to fight infection is improved. Preferably, and advantageously, de-airing of the blood is carried out automatically.

The method may comprise leaving the withdrawn blood in the reservoir for sufficient time to allow de-airing of the blood. The de-aired blood may then be returned either directly to the subject, or into a primary CPB system.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

FIG. 1 shows a schematic drawing of a cardiopulmonary bypass system (CPB) consisting of the primary artificial heart and lung support systemic system, and the secondary low pressure suction (LPS) and vent system;

FIG. 2 shows photographs of blood in a container. FIGS. 2(a) and 2(b) show well-managed suction/vent LPS blood (no air interface/mixing) using apparatus according to an embodiment of the invention, and FIGS. 2(c) and 2(d) show poorly managed suction/vent LPS blood (air interface/mixing) using a prior art LPS apparatus;

FIG. 3 shows the various components of the primary and secondary systems of one embodiment of a CPB apparatus of the invention;

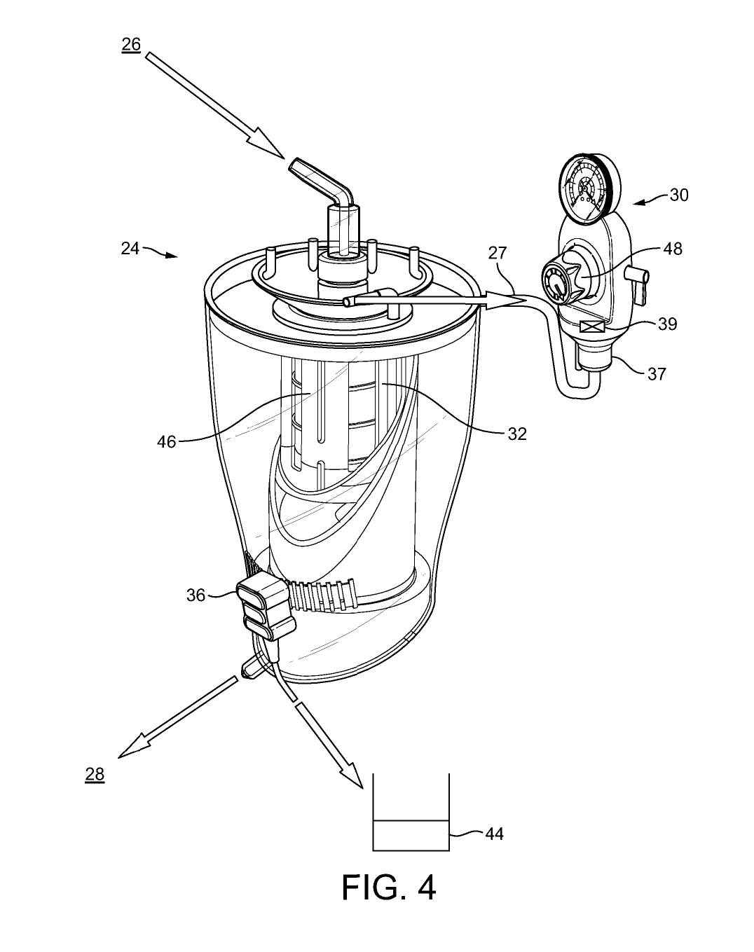

FIG. 4 shows an enlarged view of a reservoir that may be used in the apparatus of the invention;

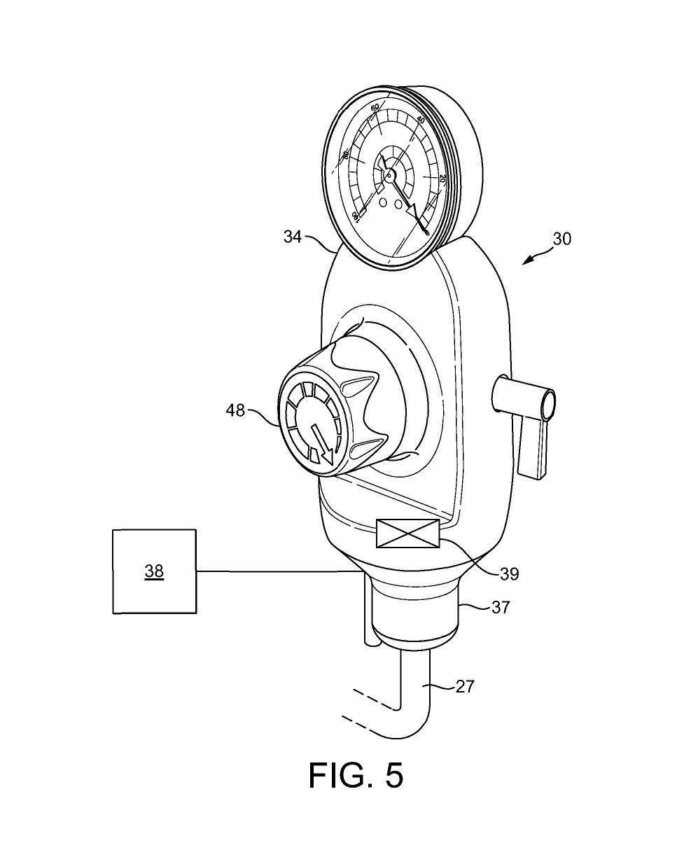

FIG. 5 shows an enlarged view of a suction regulator used in the apparatus of the invention;

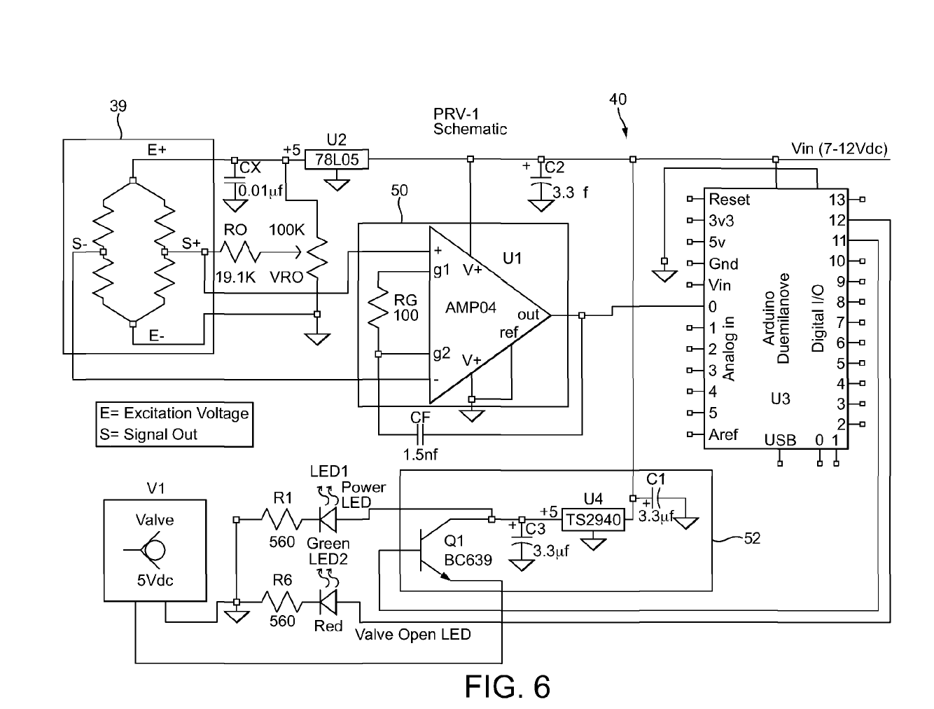

FIG. 6 shows a circuit diagram for the apparatus of the invention;

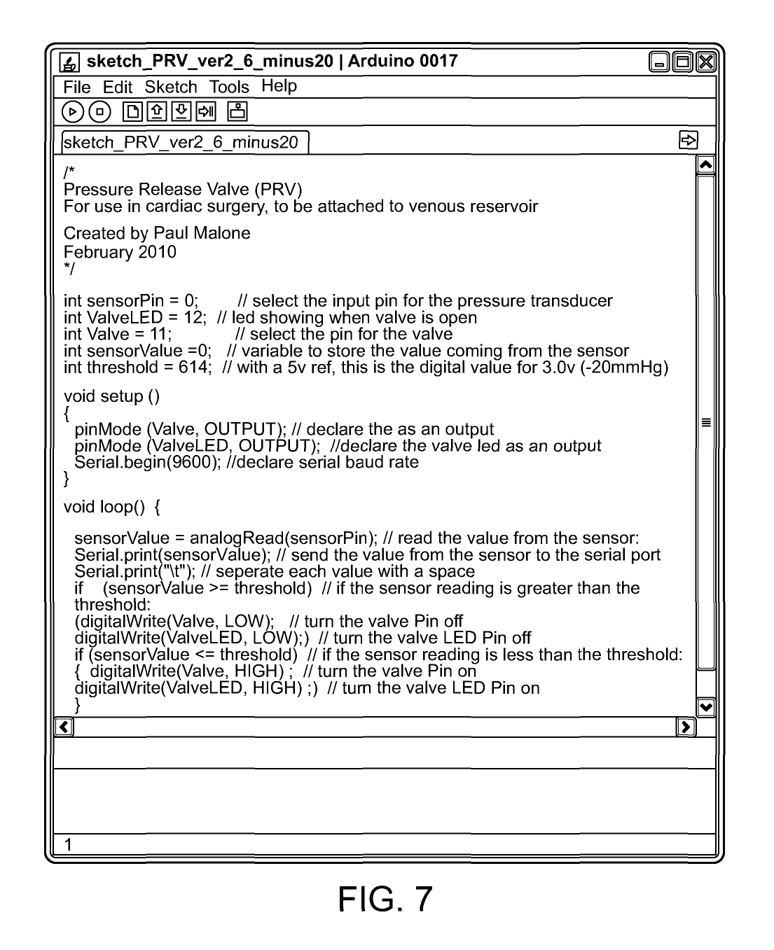

FIG. 7 shows one embodiment of an algorithm for use in controlling an automatic pressure release valve (PRV), which is located inside the suction regulator shown in FIG. 5; and

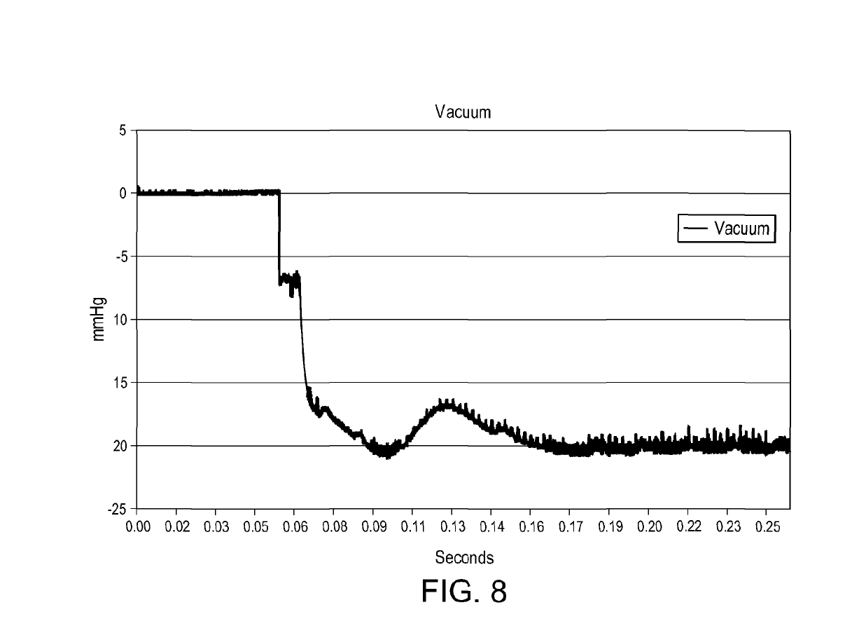

FIG. 8 shows data generated by the automatic pressure release vacuum control valve. The data show the control of the pressure inside the LPS reservoir shown in FIG. 4 in the secondary system.

### EXAMPLES

Table 1 below provides a summary of the various disadvantages that are associated with existing prior art cardiopulmonary bypass (CPB) systems, and also shows the advantages of the CPB system of the invention.

TABLE 1
Summary of CPB systems

CPB system

| | |
|---|---|
| | Drawback |
| (A) Hard shell | 1) High blood/air interface/mixing due to the surface area |
| reservoir in | of the primary reservoir, thereby causing damage. |
| series | 2) High blood non-physiological equipment exposure on |
| | every blood cycle with all blood. |
| | 3) Late in-cycle LPS/Vent blood de-airing. |
| (B) Soft shell | 1) The blood/air interface/mixing is marginally lower that |
| reservoir in | in hard-shell CPB system (A). |
| series | 2) The non-physiological equipment exposure is slightly |
| | reduced slightly compared to hard-shell CPB system (A), |
| | but still non-physiological exposure of all blood through |
| | every cycle. |
| | 3) Late LPS/Vent de-airing the same as in the hardoshall |
| | CPB system (A). |
| | 4) Have to manually de-air which takes time and an extra |
| | clinician. |
| (C) Hard shell | 1) Blood air interface as with Hard Series CPB system |
| reservoir in | (A), but not all blood on every cycle, just the LPS/Vent. |
| parallel | 2) There is still blood, non-physiological equipment |
| | exposure, but not on every cycle, just the LPS/Vent |
| | blood. |
| | 3) Earlier blood de-airing compared to systems (A) and |
| | (B) above, but not as quickly as the apparatus of the |
| | invention. |
| | 4) One has to manually return LPS/Vent blood to the |
| | primary system. |
| (D) Soft shell | 1) Reduced blood air interface experience compared |
| reservoir in | to (C). |
| parallel | 2) Reduced blood non-physiological equipment exposure |
| | compared to (C). |
| | 3) Same as (C) above. |
| | 4) One has to manually return LPS/Vent blood. |
| | 5) One has to manually de-air blood which takes time |
| | and an extra clinician. |
| | Advantages |
| INVENTION | 1) The blood is de-aired sooner in the CPB blood |
| | pathway than compared to any of systems (A), (B), |
| | (C) or (D). |
| | 2) Automatic de-airing, and so no extra clinician is |
| | needed. |
| | 3) Automatically returns the LPS/Vent blood to the |
| | primary system. |
| | 4) One can return the LPS/Vent blood to the primary |
| | system after the primary reservoir, thus reducing |
| | blood air interface. |
| | 5) It reduces blood non-physiological equipment |
| | exposure. |

Referring to FIG. 1, there is shown the layout of a conventional prior art cardiopulmonary bypass (CPB) system 2 consisting of a primary system 4 and a secondary system 6. The primary system 4 forms the artificial heart and lung support in which the patient's blood is first pumped, by a roller pump 12, out of the right atrium of the heart 8 and then eventually returned back into the left ventricle of the heart 18 following a series of treatments, as follows. From the right atrium, the blood is initially fed into a venous reservoir 10 (in series) where it is filtered and potentially mixed with fluids and drugs, if necessary. The blood is then passed to a heat exchanger 14 which allows the patient's core temperature to be varied, as necessary. The heated/cooled blood is then fed to an oxygenator 16 where oxygen is added to the blood and carbon dioxide is removed, prior to the blood being ultimately returned to the heart 18.

The secondary system 6 of the prior art CPB 2 shown in FIG. 1 is known as the low pressure suction and vent system (LPS), and involves the removal, by a roller pump 20, of blood from the patient's chest cavity, and is required to keep the operating field clear of blood to improve visibility for the surgeon during the operation. The removed blood is passed along a conduit, by the pump 20, away from the operating field, and then fed into the venous reservoir 10 of the primary system 4. Problems associated with this prior art secondary system 6 are that it involves the blood being pumped by the roller pump 20 along several non-physiological geometries, and lengths of silicone and PVC tubing, and so is very damaging to the patient's blood. The secondary system exposes the patient's blood to severe negative pressures, and also results in the patient's blood interfacing and mixing with air, thereby creating frothy blood, as shown in FIGS. 2(c) and 2(d). As discussed in the following examples, the object of the apparatus of the invention, is to avoid, or at least minimize, damage that is caused to the blood that is removed in the secondary system of CPB prior to it being fed into the venous reservoir 10 of the primary system 4.

### Example 1

With reference to FIG. 3, there is shown the layout of a “closed” CPB system 22 according to one embodiment of the invention. However, it will be appreciated that the invention also extends to a “conventional” CPB apparatus 2, as shown in FIG. 1, except with the modifications made to the secondary system 6, as described below.

The CPB system 22 shown in FIG. 3 includes a primary system 4, which is as described above in relation to known CPB systems, except that it also includes a soft shell venous blood reservoir 10 in parallel, which receives blood from a venous cannula 60. The blood is then passed, from this reservoir 10, to a primary system air removal device 62, and then on to a centrifugal arterial blood pump 64, which pumps the blood to a heat/gas exchange unit 66. From here, the blood is passed to an arterial line filter 68, and eventually back to the heart 18.

In addition to the primary system 4, the CPB system 22 of the invention shown in FIG. 3 also includes a modified and improved secondary, LPS system 6 for scavenging the blood out of the operating field or pericardium of the patient. The secondary system 2 is therefore known as a pericardial blood suction apparatus. As shown in FIG. 3, unlike in prior art CPB systems, the secondary system 6 of the invention includes a small, hard-shelled blood management reservoir 24 having a volume of approximately 10-20 Litres. The blood management reservoir 24 is shown in more detail in FIG. 4, and includes an inlet 26 through which blood withdrawn from the patient's pericardium is fed into an interconnected internal chamber 46, where it is temporarily retained and de-aired. The reservoir 24 also includes an outlet 28 through which de-aired blood exits the reservoir 24 as it is pumped, by roller pump 20, back into the primary CPB system 4. Alternatively, the de-aired blood can be fed to a holding reservoir 44.

A suction or vacuum source 30, which is shown in more detail in FIG. 5, is connected to the blood management reservoir 24 by a conduit 27. The vacuum source 30 is provided to create a gentle vacuum (i.e. a negative pressure) of about −5 to −20 mm Mercury, as indicated by vacuum gauge dial 34, which is sufficient to suck the blood from a patient's open chest cavity and into the reservoir 24 though inlet 26. Once sucked into the reservoir 24, the withdrawn blood is then allowed to temporarily settle in chamber 46 so that air can escape. The inventors have found that effective de-airing of the withdrawn blood occurs automatically inside the reservoir 24 after about 10-15 seconds. For example, as shown clearly in FIGS. 2(a) and 2(b), no froth forms in the blood, which is in contrast to the frothy condition of the blood found within the prior art LPS system, as shown in FIGS. 2(c) and 2(d).

As shown in FIG. 4, the reservoir 24 includes a blood level sensor 32, which ensures that the roller pump 20 of the LPS system automatically returns the de-aired blood from the reservoir 24 back into the primary system 4 along outlet 28. The inventors were surprised to observe the quality of the blood being returned from the reservoir 24 back into the primary system 4. Indeed, they found that allowing the blood to become de-aired in the reservoir 24 in the secondary system 6 prevented the blood from being damaged. The reservoir 24 results in a reduction in the level of mixing between the blood and air, and also a significant decrease in the level of exposure of the blood to excessive negative pressures. As a result, there is a reduction in damage caused to the red and white blood cells, and platelets, and so the blood's capacity for carrying oxygen and causing clots increases. The combined result of the condition of the blood is that the patient is in a better position to fight infection.

### Example 2

While using the apparatus 22 described in Example 1, the inventors noticed that both the roller pump 20 and the vacuum source 30 sometimes had a tendency to create a small negative pressure inside the reservoir 24 and that, accordingly, the roller pump 20 was activated when the suction inside the reservoir 24 was increased to above a desired ‘set level’. As a result, the blood inside the reservoir 24 was intermittently exposed to an increased negative pressure, which increased the amount of interface and mixing with air, and the inventors believed that this increased at least the potential of damage to the blood.

The inventors therefore carried out some modifications to the secondary LPS system 4 described in Example 1 in an attempt to further reduce the extent of damage that could be caused to the blood. The inventors have developed a mechanism for accurately controlling the vacuum that is created within the blood management reservoir 24 by the vacuum source 30 by monitoring the pressure within chamber 46, and automatically regulating it around a desired level, which is preset by the Perfusionist. The modifications that were made to the apparatus 22 include the provision of a solenoid valve 37 and a pressure transducer 39, both of which are fitted to the vacuum source 30, as shown in FIGS. 4 and 5. The apparatus 22 also includes a blood level controller 36, which controls the roller pump 20 depending on the level of blood in the reservoir 24, which is detected by level sensor 32.

The pressure transducer 39 provided on the vacuum source 30 measures the pressure inside chamber 46 within the reservoir 24. FIG. 6 illustrates a circuit diagram 40 having a pressure transducer 39, an amplifier chip 50 and a transistor switching circuit 52. Output from this circuit diagram 40 is fed, as an input signal, to an analogue input of a microprocessor 38. The code written for the microprocessor 38 then interprets the input signal and, depending on the pressure level inside the reservoir 24, latches a digital output to open or close the solenoid valve 37. Further details of the code are described below with reference to FIG. 7. Opening the solenoid valve 37 allows the internal pressure in the reservoir 24 to be equalised with atmospheric pressure until the internal pressure equals the desired level, preset by the Perfusionist. The small transistor switching circuit 52 in between the microprocessor 38 and the solenoid valve 37 supplies additional current required to open the valve 37. When the pressure inside the reservoir 24 reaches its preset level, however, the solenoid valve 37 is then closed. This process of opening and closing the solenoid valve 37 loops continually and automatically, and ensures that the pressure in the reservoir 24 is maintained about the preset fixed point.

The control loop continues until such time that the apparatus 22 is switched off, and blood is no longer drawn from the patient. The apparatus 22 is able to auto-calibrate the pressure transducer 39 as it is switched on, and has a switch 48 by which the perfusionist can adjust the set-point level of the vacuum at which point the valve 37 opens. The apparatus 22 is either mains or battery operated.

In summary, the apparatus 22 consists of three main stages, i.e. the input stage, the signal processing stage and the output stage.

Input Stage:

The input stage consists of the pressure transducer 39 and a strain gauge amplifier, which amplifies the signal coming from the transducer 39 and feeds a 0-5V signal to the signal processing stage carried out by the microprocessor 38.

Signal Processing Stage:

This stage consists of an ‘Arduino Duemilanove’ microcontroller development board having multiple analogue inputs, and multiple digital inputs and outputs. Code has been written and downloaded onto the board for it to function, which allows inputs to be read and then outputs to be set accordingly. An analogue input takes the output from the pressure transducer 39, and when the voltage drops below a specified level, the board enables a digital output to open the solenoid valve 37. The analogue input pins provide 10 bits of resolution corresponding to 1024 different values, and, by default, they measure from ground to 5V.

The digital input/output pins are used as outputs, and can provide 40 mA current. The board is programmed using the Arduino programming language which is based on an open source programming language called ‘Wiring’ which was developed at the MIT Media Lab and Interaction Design Institute Ivrea by Ben Fry and Casey Reas. It is based on the C programming language. To use the programmer, the application software is run on a desktop PC 38 and enables code to be written in ‘Sketches’. To allow the board to communicate with a PC, USB drivers have to be installed and then the ‘sketch’ can be downloaded to the microcontroller using the Arduino application. Referring to FIG. 7, there is shown an example of a prototype ‘Sketch’.

Output Stage:

The output stage takes a digital signal (zero for off and 5V for on) from the Arduino and switches the solenoid valve 37 on or off. The valve 37 has an inlet and an outlet. The outlet is connected to the internal chamber 46 of the reservoir 24 and the inlet is open to draw in atmospheric pressure when the valve 37 opens.

### Example 3

The inventors have carried out tests to demonstrate that the pressure transducer 39 and the solenoid valve 37 can be used to efficiently maintain the pressure within the reservoir 24 at a predetermined set-point value.

Referring to FIG. 8, there is shown data generated by the automatic pressure release vacuum control valve 37. The data show the tight control of the pressure inside the LPS reservoir 24 in the secondary system 6. The graph shows pressure control within the reservoir over time. At 0.6 seconds, the suction means is turned on, thereby reducing the pressure down to the desired fixed set-point of −20 mm/mercury where the pressure is maintained. The small peak in the graph at approximately 0.13 s is explained by the wand/suction apparatus leaving the test bucket for a split second, air getting in, or pressure not being maintained (as would happen with blood pooling in the chest cavity of a patient).

In summary, the apparatus 22 of the invention overcomes the problems associated with known secondary systems in which the patient's blood is damaged prior to it being introduced into the primary system, as shown in FIGS. 2(c) and 2(d). Due to the presence of the reservoir 24, the secondary LPS system 22:
(i) reduces the exposure of the patient's blood to excessive negative pressures and air;
(ii) automates the removal of any air, which may be unavoidably drawn into the secondary LPS system; and
(iii) automates the return of the withdrawn blood into the primary CPB circuit, for example when using either a closed or conventional CPB circuit.

The reservoir 24 has a blood inlet 26 for receiving blood removed under negative pressure from the patient's pericardium, a blood outlet 28 for removing de-aired blood from the reservoir 24, and a vacuum inlet for supplying a vacuum to the reservoir 24 from the vacuum source 30.

1. A pericardial blood suction apparatus for use in cardiopulmonary bypass (CPB), wherein the apparatus comprises means for withdrawing blood from a subject's chest cavity and/or heart chamber, and a reservoir in which withdrawn blood is de-aired.

2. An apparatus according to claim 1, wherein the apparatus is a low pressure suction (LPS) and/or vent system (i.e. the secondary system) of CPB, and is capable of conserving blood and keeping the operating field of the chest cavity substantially clear of blood during an operation, and/or wherein the apparatus automatically de-airs the blood in the reservoir.

3. (canceled)

4. An apparatus according to claim 1, wherein the apparatus returns the de-aired blood either automatically or manually to the systemic (i.e. the primary) system of CPB system, or a parallel reservoir.

5. An apparatus according to claim 1, wherein the volume of the reservoir is between approximately 10 ml and 500 ml, or between 15 ml and 250 ml.

6. An apparatus according to claim 1, wherein the reservoir comprises a blood inlet through which withdrawn blood is fed into the reservoir, and a blood outlet through which de-aired blood exits the reservoir.

7. An apparatus according to claim 1, wherein the reservoir comprises an internal chamber which, in use, contains the withdrawn blood for sufficient time to allow de-airing to occur, for example between approximately 1 and 20 seconds, or between approximately 2 and 10 seconds.

8. An apparatus according to claim 1, wherein the means for withdrawing the blood from the subject comprises engagement means which is capable of engaging with the subject's chest cavity, the engagement means being in fluid communication with the blood inlet, optionally wherein the engagement means comprises a suction catheter, a cannula, a yanker sucker, a wand or the like.

9. (canceled)

10. An apparatus according to claim 1, wherein the means for withdrawing the subject's blood comprises a source of negative pressure, i.e. suction pressure.

11. An apparatus according to claim 1, wherein the means for withdrawing the blood is adapted to create a negative pressure in the reservoir of at least −1 to −120 mm Mercury, preferably −5 to −20 mm Mercury.

12. An apparatus according to claim 1, wherein the means for withdrawing the blood is a pump connected to the reservoir.

13. An apparatus according to claim 1, wherein the means for withdrawing the blood is a vacuum source.

14. An apparatus according to claim 1, wherein the apparatus comprises feed means for feeding blood from the reservoir to the subject, the primary CPB system or a holding chamber, optionally wherein the feed means comprises a pump, for example a roller pump.

15. (canceled)

16. An apparatus according to claim 14, wherein the reservoir comprises a blood sensor, which is adapted, in use, to monitor the volume of blood in the reservoir and control the feed means depending on the blood volume, optionally wherein, in use, as the volume of blood reaches an upper preset level within the reservoir, the blood sensor is capable of switching the pump on, to thereby pump blood out of the reservoir, and as the volume of blood reaches a lower preset level within the reservoir, the blood sensor is capable of switching the pump off, to thereby prevent blood from being pumped out of the reservoir.

17. (canceled)

18. An apparatus according to claim 1, wherein the pericardial blood suction apparatus comprises pressure control means adapted, in use, to control the pressure within the reservoir, and automatically regulate it about a desired preset value, optionally wherein the pressure control means comprises a pressure transducer which is adapted, in use, to measure the pressure inside the reservoir, and/or wherein the pressure transducer is connected to the means for withdrawing blood from the subject, preferably via an air inlet thereof.

19. (canceled)

20. (canceled)

21. An apparatus according to claim 18, wherein the pressure control means comprises a valve, which valve, when open, connects the reservoir to atmospheric pressure, optionally wherein the pressure control means comprises a control circuit, which is adapted to control the opening and dosing of the valve depending on the pressure within the reservoir, and/or wherein the valve is a controllable valve, such as a solenoid valve.

22. (canceled)

23. (canceled)

24. A cardiopulmonary bypass system (CPB) comprising the pericardial blood suction apparatus according to claim 1.

25. The CPB system according to claim 24, wherein the CPB system is a conventional circuit system or a closed circuit system.

26. (canceled)

27. The CPB system according to claim 24, wherein the CPB system comprises a hard-shell reservoir arranged in series or in parallel.

28. (canceled)

29. The CPB system according to claim 24, wherein the CPB system comprises a soft-shell reservoir arranged in series or in parallel.

30. (canceled)

31. Use of the pericardial blood suction apparatus according to claim 1, for reducing damage caused to blood withdrawn from a subject's pericardium and/or heart chamber.

32. Use of a cardiopulmonary bypass system (CPB) for reducing damage caused to blood withdrawn from a subject's pericardium and/or heart chamber, the cardiopulmonary bypass system comprising the pericardial blood suction apparatus according to claim 1.
